(19) United States
(12) Patent Application Publication
(10) Pub. No.: US 2024/0343663 A1
(43) Pub. Date: Oct. 17, 2024

(54) CATALYSTS FOR DEHYDROGENATION PROCESS

(71) Applicant: Dow Global Technologies LLC, Midland, MI (US)

(72) Inventors: Lin Luo, Sugarland, TX (US); Adrianus Koeken, Terneuzen (NL); Mingzhe Yu, Sugarland, TX (US); Andrzej Malek, Midland, MI (US); Hangyao Wang, Pearland, TX (US); Luis Bollmann, Pearland, TX (US)

(73) Assignee: Dow Global Technologies LLC, Midland, MI (US)

(21) Appl. No.: 18/683,693

(22) PCT Filed: Aug. 29, 2022
(86) PCT No.: PCT/US2022/041885

§ 371 Date: Feb. 14, 2024

Related U.S. Application Data
(60) Provisional application No. 63/238,940, filed on Aug. 31, 2021.

(51) Int. Cl. C07C 5/333; B01J 8/24; B01J 23/62; B01J 23/96

(52) U.S. Cl. CPC C07C 5/3337; B01J 8/24; B01J 23/62; B01J 23/96; C07C 2523/62

(57) ABSTRACT

A method for dehydrogenation of one or more hydrocarbons and regeneration and reactivation of a catalyst composition includes contacting a first gaseous stream comprising a first hydrocarbon, such as propane, with a catalyst composition in a dehydrogenation reactor at a first temperature, thereby producing a first dehydrogenated hydrocarbon, such as propylene, and a deactivated catalyst composition; combusting at least one fuel gas and coke on the deactivated catalyst in the presence of oxygen at a second temperature, thereby producing a heated catalyst composition; and reactivating the catalyst in the presence of oxygen. The second temperature is from 50° C. to 200° C. greater than the first temperature. The catalyst composition is also described and comprises gallium, platinum and a further noble metal, such as palladium.

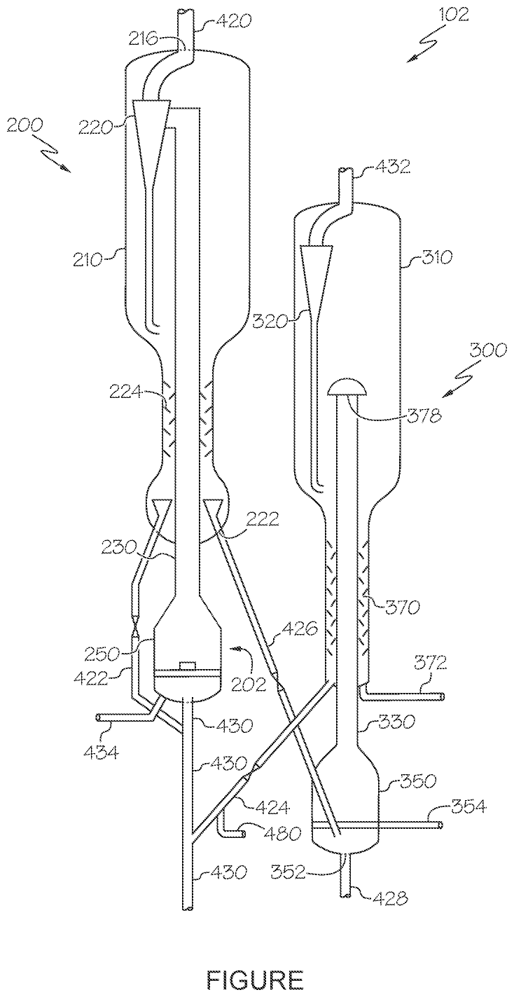

### CROSS-REFERENCE TO RELATED APPLICATIONS

The present application claims priority to U.S. Provisional Patent Application No. 63/238,940, filed Aug. 31, 2021, and entitled “CATALYSTS FOR DEHYDROGENATION PROCESS,” the entirety of which is incorporated by reference herein.

### TECHNICAL FIELD

Embodiments of the present disclosure generally relate to dehydrogenation of hydrocarbons, and in particular, methods of dehydrogenating hydrocarbons and regenerating and reactivating a dehydrogenation catalyst system.

### BACKGROUND

Light olefins, such as ethylene, may be used as base materials to produce many different materials, such as polyethylene, vinyl chloride, and ethylene oxide, which may be used in product packaging, construction, and textiles. As a result of this utility, there is a worldwide increasing demand for light olefins. Suitable processes for producing light olefins generally depend on the given chemical feed and include, for example, fluidized catalytic dehydrogenation (FCDh) processes.

### SUMMARY

Generally, in FCDh processes, a hydrocarbon-containing feed and a fluidized catalyst are introduced into a reactor portion of an FCDh system, the hydrocarbon-containing feed contacts the catalyst, and the resulting mixture flows through the reactor portion to undergo dehydrogenation, thereby producing a dehydrogenated hydrocarbon and a deactivated catalyst composition. The catalyst composition may be separated from the dehydrogenated hydrcarbon and passed to a catalyst-processing portion of the FCDh system. Typically, the heat necessary for dehydrogenation in FCDh processes is primarily provided by the combustion of a combustion fuel, such as coke deposited on the catalyst and/or a supplemental fuel, in the catalyst-processing portion. Specifically, catalyst that has been heated by the combustion of the combustion fuel in the catalyst-processing portion transfers heat to the reactor portion. In order to combust the combustion fuel at reasonable temperatures, the catalyst is relied upon to provide combustion activity. An efficient FCDh system would allow for the rapid change of products via a change in the composition of the hydrocarbon-containing feed. However, the composition of the feed may affect the amount of heat required to perform the dehydrogenation. For instance, to attain a 50% conversion of the respective feed, isobutane dehydrogenation requires a temperature of about 570° C., propane dehydrogenation requires a temperature of about 630° C., and ethane dehydrogenation requires a temperature of about 770° C., using isothermal conditions for ease of comparison. The catalyst systems and methods for dehydrogenating hydrocarbons of the present disclosure may increase operational flexibility of a reactor system, including the catalyst used therein, so that the dehydrogenation of various feeds may be accomplished using the same reactor system. This is accomplished, at least in part, by the utilization of catalysts described herein, which include gallium, platinum, and at least one other noble metal.

According to aspects, a method for dehydrogenation of one or more hydrocarbons and regeneration and reactivation of a catalyst composition includes contacting a first gaseous stream comprising a first hydrocarbon with a catalyst composition in a dehydrogenation reactor at a first temperature, thereby producing a first dehydrogenated hydrocarbon and a deactivated catalyst composition; combusting at least one fuel gas and coke on the deactivated catalyst in the presence of oxygen at a second temperature, thereby producing a heated catalyst composition; and reactivating the catalyst in the presence of oxygen. The second temperature is from 50° C. to 200° C. greater than the first temperature. The catalyst composition includes an active metal comprising gallium, a support, and a promoter comprising platinum and at least one noble metal selected from the group consisting of ruthenium, rhodium, palladium, rhenium, iridium, and a combination of two or more thereof. The ratio of total second noble metal to platinum by weight is from 0.05 to 1.5.

According to aspects, a catalyst composition includes an active metal comprising gallium, a support, and a promoter comprising platinum and at least one noble metal selected from the group consisting of ruthenium, rhodium, palladium, rhenium, iridium, and a combination of two or more thereof. The ratio of total second noble metal to platinum by weight is from 0.05 to 1.5.

It has been found that the ability to vary the feedstock for a dehydrogenation reactor is enhanced when using a dehydrogenation catalyst composition comprising an active metal composition and a promoter, where the promoter includes platinum and at least one additional noble metal. The ability to vary the feedstock may be further enhanced by including the additional noble metal and platinum in an additional noble metal-to-platinum weight ratio from 0.05 to 1.5. Additionally, the catalyst compositions described herein allow for regeneration of the catalyst composition at lower temperatures. As a result, the regeneration may be conducted at a temperature that is from 50° C. to 200° C. greater than the temperature at which the dehydrogenation is conducted. This, in turn, may further enhance the ability to vary the feedstock.

It is to be understood that both the foregoing brief summary and the following detailed description present embodiments of the technology, and are intended to provide an overview or framework for understanding the nature and character of the technology as it is claimed. The accompanying drawings are included to provide a further understanding of the technology, and are incorporated into and constitute a part of this specification. The drawings illustrate various embodiments and, together with the description, serve to explain the principles and operations of the technology. Additionally, the drawings and descriptions are meant to be merely illustrative, and are not intended to limit the scope of the claims in any manner.

Additional features and advantages of the described embodiments will be set forth in the detailed description that follows. The additional features and advantages of the described embodiments will be, in part, readily apparent to those skilled in the art from that description or recognized by practicing the described embodiments, including the detailed description that follows as well as the drawings and the claims.

### BRIEF DESCRIPTION OF THE DRAWINGS

The following detailed description of specific embodiments of the present disclosure can be best understood when read in conjunction with the following drawings, where like structure is indicated with like reference numerals and in which:

The FIGURE schematically depicts a reactor system, according to one or more embodiments of the present disclosure.

Reference will now be made in greater detail to various embodiments, some of which are illustrated in the accompanying drawing.

### DETAILED DESCRIPTION

According to one or more embodiments described herein, methods and catalysts may be used for dehydrogenation of hydrocarbon-containing feedstocks using, for instance, fluidized reactor systems. The catalysts may become deactivated and require regeneration and reactivation, including combustion of fuel gas and/or coke deposits on the catalysts. The various embodiments will now be discussed in more detail.

As used in the present disclosure, the term “fluidized reactor system” refers to a reactor system in which one or more reactants are contacted with a catalyst in a fluidization regime, such as bubbling regime, slug flow regime, turbulent regime, fast fluidization regime, pneumatic conveying regime, or combinations of these, in different portions of the system. For example, in a fluidized reactor system, a chemical feed containing one or more reactants may be contacted with the circulating catalyst at an operating temperature to conduct a continuous reaction to produce an effluent.

As used in the present disclosure, the term “deactivated catalyst” or “spent catalyst” refers to a catalyst having decreased catalytic activity resulting from buildup of coke and/or loss of catalyst active sites. The terms “catalytic activity” and “catalyst activity” refer to the degree to which the catalyst is able to catalyze the reactions conducted in the reactor system.

As used in the present disclosure, the terms “catalyst reactivation” and “reactivating the catalyst” refer to processing the deactivated catalyst to restore at least a portion of the catalyst activity to produce a reactivated catalyst. The deactivated catalyst may be reactivated by, but not limited to, recovering catalyst acidity, oxidizing the catalyst, other reactivation process, or combinations thereof.

As noted above, the heat and temperature requirements for the dehydrogenation are dependent, at least in part, on the predominant hydrocarbon in the gaseous hydrocarbon-containing stream. For example, the reaction heat needed for dehydrogenation of isobutane is about 15% lower than that required for ethane dehydrogenation, and the reaction temperature is about 200° C. lower. When switching feedstock from predominantly propane to predominantly isobutane, the reaction heat needed for dehydrogenation of isobutane is about 6% lower than that required for propane dehydrogenation, while the reaction temperature is about 60° C. lower. Thus, when switching dehydrogenation feedstocks (e.g. from ethane to isobutane or from propane to isobutane), significant adjustment is needed to allow matching of the required reaction temperature and reaction heat.

The reaction heat needed per unit time can be expressed as a function of catalyst circulation rate and delta T between regeneration and reaction, as provided in Equation (1):

$$\\ \begin{matrix}\frac{F \times Δ {H}_{rxn}}{Δ t} = \frac{{Q}_{Regen \to Reactor}}{Δ t} = {R}_{Cat} \times {C}_{P , Cat} \times ( {T}_{Regen} - {T}_{Reactor} ) & ( 1 )\end{matrix}$$

where F is the reactant (ethane, propane or butane) molar flow rate, ΔH_{r×n} is the molar heat of the dehydrogenation reaction taking place in the reactor, A/is the unit time, Q_{Regen→Reactor} is the heat carried over from the regenerator to the reactor, R_{Cat} is the catalyst circulation rate, C_{P,Cat} is the heat capacity of the catalyst solid, T_{Regen} is the catalyst temperature at the outlet of regenerator and T_{Reactor} is the catalyst temperature at the outlet of reactor. The heat capacity of the catalyst solid is approximately constant within the temperature ranges of interest herein.

One manner to make minor adjustments to meet the requirement for reaction temperature and heat is to change the catalyst circulation rate from the regenerator to reactor. If the catalyst circulation rate is the only parameter to be adjusted, however, it is difficult to maintain the correct reactor temperature (T_{Reactor}) while also maintaining the appropriate heat of the reaction (Equation (1)). For example, when switching from propane to isobutane as the dehydrogenation feedstock, keeping the molar flow rate of hydrocarbons the same and T_{Regen} at constant 750° C., T_{Reactor} for propane is 630° C. and T_{Regen}−T_{Reactor} is 120° C. However, for isobutane, the T_{Reactor} is 570° C. and T_{Regen}−T_{Reactor} is 180° C. Thus, to meet the heat and temperature requirements when changing the feedstock from propane to isobutane, the catalyst circulation rate needs to be reduced by about 40%.

For a fixed design, there is a limit on how much the catalyst circulation rate may be adjusted. This catalyst circulation rate is further limited by the range of catalyst to feed ratio needed to provide sufficient catalyst activity for the dehydrogenation. Additionally, the molar flow rate of the reactants is not an independent parameter due to the requirement of proper hydrodynamics.

The regenerator temperature (T_{Regen}) may be adjusted to help meet the reaction temperature and reaction heat criteria. For example, T_{Regen} may be adjusted by changing the amount of fuel gas injected into the regenerator vessel for combustion, which is discussed further below. However, when reducing the amount of fuel gas for combustion, the temperature for the catalyst in the combustion zone is also reduced, sometimes to such a degree that the temperature is too low for complete, or nearly complete, fuel gas combustion. This may be especially troublesome when CH₄ based fuel gas is used, because the amount of unreacted CH₄ in the effluent may be higher than the Lower Flammable Limit, thereby presenting significant safety risks.

Therefore, when switching dehydrogenation feedstocks, careful consideration is needed for many parameters, including reaction heat requirement, catalyst circulation rate, reactant feed flow rate, reaction temperature, and regeneration temperature. The methods and catalysts described herein help to simplify the selection of these criteria by providing the ability to perform fuel gas combustion over a broad range of temperatures, thereby allowing simplified adjustment of T_{Regen}. As a result, the methods and compositions disclosed herein allow control of the ability to meet the requirements of the reaction temperature, the reaction heat, and the amount of catalyst needed for dehydrogenation of different feedstocks.

The catalyst systems and methods for producing dehydrogenated hydrocarbons of the present disclosure will now be described in the context of an example FCDh system. It should be understood that the schematic diagram of the FIGURE is only an example system and that other FCDh systems are contemplated as well, and the concepts described may be utilized in such alternate systems. For example, the concepts described may be equally applied to other systems with alternate reactor units and regeneration units, such as those that operate under non-fluidized conditions or are downers rather than risers. Additionally, the presently described catalyst systems and methods for producing dehydrogenated hydrocarbons should not be limited only to embodiments for reactor systems designed to produce light olefins through FCDh processes, such as the reactor system described with respect to the FIGURE, as other dehydrogenation systems (e.g., utilizing different chemical feeds) are contemplated. When describing the simplified schematic illustration of the FIGURE, the numerous valves, temperature sensors, electronic controllers, and the like, which may be used and are well known to a person of ordinary skill in the art, are not included. Further, accompanying components that are often included in such reactor systems, such as air supplies, heat exchangers, surge tanks, and the like are also not included. However, it should be understood that these components are within the scope of the present disclosure.

Referring now to the FIGURE, an example reactor system 102 is schematically depicted. The reactor system 102 generally includes a reactor portion 200 and a catalyst-processing portion 300. As used in the context of the FIGURE, the reactor portion 200 refers to the portion of the reactor system 102 in which the major process reaction takes place. For example, the reactor system 102 may be an FCDh system in which a hydrocarbon-containing feed is dehydrogenated in the presence of a dehydrogenation catalyst in the reactor portion 200 of the reactor system 102. The reactor portion 200 generally includes a reactor 202, which may include an upstream reactor section 250, a downstream reactor section 230, and a catalyst separation section 210, which serves to separate catalyst from effluent produced in the reactor 202.

Similarly, as used in the context of the FIGURE, the catalyst-processing portion 300 refers to the portion of the reactor system 102 in which catalyst is processed in some way, such as removal of coke deposits, heating, reactivating, or combinations of these. The catalyst-processing portion 300 generally includes a combustor 350, a riser 330, a catalyst separation section 310, and an oxygen treatment zone 370. The combustor 350 may be in fluid communication with the riser 330. The combustor 350 may also be in fluid communication with the catalyst separation section 210 via standpipe 426, which may supply deactivated catalyst from the reactor portion 200 to the catalyst processing portion 300 for catalyst processing (e.g., coke removal, heating, reactivating, etc.). The oxygen treatment zone 370 may be in fluid communication with the upstream reactor section 250 (e.g., via standpipe 424 and transport riser 430), which may supply processed catalyst from the catalyst processing portion 300 back to the reactor portion 200. The combustor 350 may include one or more lower combustor inlet ports 352 where air inlet 428 connects to the combustor 350. The air inlet 428 may supply air and/or other reactive gases, such as an oxygen-containing gas to the combustor 350. The combustor 350 may also include a fuel inlet 354, which may supply a fuel, such as a hydrocarbon stream, to the combustor 350. The oxygen treatment zone 370 may include an oxygen-containing gas inlet 372, which may supply an oxygen-containing gas to the oxygen treatment zone 370 for oxygen treatment of the catalyst.

Referring still to the FIGURE, general operation of the reactor system 102 to conduct a dehydrogenation reaction under normal operating conditions will be described. During operation of the reactor portion 200 of the reactor system 102, a hydrocarbon-containing feed may enter the reactor portion 200 via feed inlet 434 and contact a fluidized catalyst introduced to the reactor portion 200 via a transport riser 430 and a dehydrogenated hydrocarbon effluent may exit the reactor portion 200 via pipe 420. In one or more embodiments, the hydrocarbon-containing feed and a fluidized catalyst are introduced into the upstream reactor section 250, the hydrocarbon-containing feed contacts the catalyst in the upstream reactor section 250, and the resulting mixture flows upwardly into and through the downstream reactor section 230 to produce the olefin-containing effluents.

In one or more embodiments, the hydrocarbon-containing feed includes ethane, propane, n-butane, i-butane, ethylbenzene, or combinations of these. In some embodiments, the hydrocarbon-containing feed includes at least 50 weight percent (wt. %), at least 60 wt. %, at least 70 wt. %, at least 80 wt. %, at least 90 wt. %, at least 95 wt. %, or at least 99 wt. % ethane. In some embodiments, the hydrocarbon-containing feed includes at least 50 wt. %, at least 60 wt. %, at least 70 wt. %, at least 80 wt. %, at least 90 wt. %, at least 95 wt. %, or at least 99 wt. % propane. In some embodiments, the hydrocarbon-containing feed includes at least 50 wt. %, at least 60 wt. %, at least 70 wt. %, at least 80 wt. %, at least 90 wt. %, at least 95 wt. %, or at least 99 wt. % of n-butane. In some embodiments, the hydrocarbon-containing feed includes at least 50 wt. %, at least 60 wt. %, at least 70 wt. %, at least 80 wt. %, at least 90 wt. %, at least 95 wt. %, or at least 99 wt. % of i-butane. In some embodiments, the hydrocarbon-containing feed includes at least 50 wt. %, at least 60 wt. %, at least 70 wt. %, at least 80 wt. %, at least 90 wt. %, at least 95 wt. %, or at least 99 wt. % of ethylbenzene. In some embodiments, the hydrocarbon-containing feed includes at least 50 wt. %, at least 60 wt. %, at least 70 wt. %, at least 80 wt. %, at least 90 wt. %, at least 95 wt. %, or at least 99 wt. % of the sum of ethane, propane, n-butane, i-butane, and ethylbenzene.

The temperature at which the reactor portion 200 of the reactor system 102 is operated may depend upon the species of hydrocarbon being dehydrogenated. For instance, in embodiments, the hydrocarbon undergoing dehydrogenation may comprise ethane and the temperature at which the dehydrogenation is conducted may be from 700° C. to 850° C., such as from 710° C. to 850° C., from 720° C. to 850° C., from 730° C. to 850° C., from 740° C. to 850° C., from 750° C. to 850° C., from 760° C. to 850° C., from 770° C. to 850° C., from 780° C. to 850° C., from 790° C. to 850° C., from 800° C. to 850° C., from 810° C. to 850° C., from 820° C. to 850° C., from 830° C. to 850° C., from 840° C. to 850° C., from 700° C. to 840° C., from 700° C. to 830° C., from 700° C. to 820° C., from 700° C. to 810° C., from 700° C. to 800° C., from 700° C. to 790° C., from 700° C. to 780° C., from 700° C. to 770° C., from 700° C. to 760° C., from 700° C. to 750° C., from 700° C. to 740° C., from 700° C. to 730° C., from 700° C. to 720° C., or from 700° C. to 710° C.

In embodiments, the hydrocarbon undergoing dehydrogenation may comprise propane and the temperature at which the dehydrogenation is conducted may be from 550° C. to 700° C., such as from 560° C. to 700° C., from 570° C. to 700° C., from 580° C. to 700° C., from 590° C. to 700° C., from 600° C. to 700° C., from 610° C. to 700° C., from 620° C. to 700° C., from 630° C. to 700° C., from 640° C. to 700° C., from 650° C. to 700° C., from 660° C. to 700° C., from 670° C. to 700° C., from 680° C. to 700° C., from 690° C. to 700° C., from 550° C. to 690° C., from 550° C. to 680° C., from 550° C. to 670° C., from 550° C. to 660° C., from 550° C. to 650° C., from 550° C. to 640° C., from 550° C. to 630° C., from 550° C. to 620° C., from 550° C. to 610° C., from 550° C. to 600° C., from 550° C. to 590° C., from 550° C. to 580° C., from 550° C. to 570° C., or from 550° C. to 560° C.

In embodiments, the hydrocarbon undergoing dehydrogenation may comprise isobutane and the temperature at which the dehydrogenation is conducted may be from 500° C. to 650° C., such as from 510° C. to 650° C., from 520° C. to 650° C., from 530° C. to 650° C., from 540° C. to 650° C., from 550° C. to 650° C., from 560° C. to 650° C., from 570° C. to 650° C., from 580° C. to 650° C., from 590° C. to 650° C., from 600° C. to 650° C., from 610° C. to 650° C., from 620° C. to 650° C., from 630° C. to 650° C., from 640° C. to 650° C., from 500° C. to 640° C., from 500° C. to 630° C., from 500° C. to 620° C., from 500° C. to 610° C., from 500° C. to 600° C., from 500° C. to 590° C., from 500° C. to 580° C., from 500° C. to 570° C., from 500° C. to 560° C., from 500° C. to 550° C., from 500° C. to 540° C., from 500° C. to 530° C., from 500° C. to 520° C., or from 500° C. to 510° C.

In one or more embodiments, the dehydrogenated hydrocarbon effluent includes light olefins. As used in the present disclosure, the term “light olefins” refers to one or more of ethylene, propylene, and butene. The term butene includes any isomers of butene, such as α-butylene, cis-β-butylene, trans-β-butylene, and isobutylene. In some embodiments, the dehydrogenated hydrocarbon effluent includes at least 25 wt. % light olefins based on the total weight of the dehydrogenated hydrocarbon effluent. For example, the dehydrogenated hydrocarbon effluent may include at least 35 wt. % light olefins, at least 45 wt. % light olefins, at least 55 wt. % light olefins, at least 65 wt. % light olefins, or at least 75 wt. % light olefins based on the total weight of the dehydrogenated hydrocarbon effluent.

In one or more embodiments, the catalyst includes an active metal component, a promoter component including platinum and at least one other noble metal, and a support. In embodiments, the active metal component comprises gallium. In embodiments, the active metal component consists of gallium.

In one or more embodiments, the catalyst includes from 0.1 wt. % to 10 wt. % active metal component based on the total weight of the catalyst. For example, the catalyst may include from 0.1 wt. % to 7.5 wt. %, from 0.1 wt. % to 5 wt. %, from 0.1 wt. % to 2.5 wt. %, from 0.1 wt. % to 0.5 wt. %, from 0.5 wt. % to 10.0 wt. %, from 0.5 wt. % to 7.5 wt. %, from 0.5 wt. % to 5 wt. %, from 0.5 wt. % to 2.5 wt. %, from 2.5 wt. % to 10.0 wt. %, from 2.5 wt. % to 7.5 wt. %, from 2.5 wt. % to 5 wt. %, from 5 wt. % to 10 wt. %, from 5 wt. % to 7.5 wt. %, or from 7.5 wt. % to 10 wt. % active metal component based on the total weight of the catalyst. Without intending to be bound by any particular theory, it is believed that a catalyst containing less than 0.1 wt. % active metal component may not provide sufficient or commercially viable dehydrogenation activity. Further, it is believed that a catalyst containing more than 10 wt. % active metal may not provide enough additional dehydrogenation activity to justify the increased cost of including a greater amount of active metal.

In one or more embodiments, the catalyst includes a promoter component including from 5 ppmw to 500 ppmw platinum based on the total weight of the catalyst. For example, the catalyst may include from 5 ppmw to 450 ppmw, from 5 ppmw to 400 ppmw, from 5 ppmw to 350 ppmw, from 5 ppmw to 300 ppmw, from 5 ppmw to 250 ppmw, from 5 ppmw to 200 ppmw, from 5 ppmw to 150 ppmw, from 5 ppmw to 100 ppmw, from 5 ppmw to 50 ppmw, from 50 ppmw to 500 ppmw, from 100 ppmw to 500 ppmw, from 150 ppmw to 500 ppmw, from 200 ppmw to 500 ppmw, from 250 ppmw to 500 ppmw, from 300 ppmw to 500 ppmw, from 350 ppmw to 500 ppmw, from 400 ppmw to 500 ppmw, or from 450 ppmw to 500 ppmw platinum based on the total weight of the catalyst.

In one or more embodiments, the catalyst includes a ratio of active metal to platinum by weight from 5 to 600. For example, the ratio of active metal to platinum by weight may be from 5 to 550, from 5 to 500, from 5 to 450, from 5 to 400, from 5 to 350, from 5 to 300 from 5 to 250, from 5 to 200, from 5 to 150, from 5 to 100, from 5 to 50, from 5 to 10, from 10 to 600, from 50 to 600, from 100 to 600, from 150 to 600, from 200 to 600, from 50 to 600, from 300 to 600, from 350 to 600, from 400 to 600, from 450 to 600, from 500 to 600, from 550 to 600, or even from 590 to 600. Without intending to be bound by any particular theory, it is believed that a catalyst containing a ratio of active metal to platinum by weight less than 5 may not provide the desired dehydrogenation activity. Further, it is believed that a catalyst containing a ratio of active metal to platinum by weight greater than 600 may not be able to be sufficiently reactivated and/or may not demonstrate the desired selectivity.

The promoter component of the catalyst further includes a second noble metal. The second noble metal may be ruthenium, rhodium, palladium, rhenium, iridium, or a combination of two or more thereof. In embodiments, the second noble metal is palladium. Without intending to be bound by any particular theory, it is believed that the presence of this second noble metal may improve the ability of the catalyst to combust the combustion fuel, as described below, such that lower temperatures may be used during the catalyst processing stage after the dehydrogenation stage. In this way, a lower temperature may be used for processing the deactivated catalyst after the dehydrogenation relative to the temperatures needed when the promoter component contains platinum but no second noble metal.

In one or more embodiments, the catalyst includes a ratio of second noble metal to platinum by weight is from 0.05 to 1.5. For example, the ratio of second noble metal to platinum by weight is from 0.05 to 1.4, from 0.05 to 1.3, from 0.05 to 1.2, from 0.05 to 1.1, from 0.05 to 1, from 0.05 to 0.9, from 0.05 to 0.8, from 0.05 to 0.7, from 0.05 to 0.6, from 0.05 to 0.5, from 0.05 to 0.4, from 0.05 to 0.3, from 0.05 to 0.2, from 0.05 to 0.1, from 0.1 to 1.5, from 0.2 to 1.5, from 0.3 to 1.5, from 0.4 to 1.5, from 0.5 to 1.5, from 0.6 to 1.5, from 0.7 to 1.5, from 0.8 to 1.5, from 0.9 to 1.5, from 1 to 1.5, from 1.1 to 1.5, from 1.2 to 1.5, from 1.3 to 1.5, or from 1.4 to 1.5.

In one or more embodiments, the catalyst optionally includes a second promoter selected from the group consisting of an alkali metal, an alkaline earth metal, and a combination of the alkali metal and the alkaline earth metal. In one or more embodiments, the catalyst composition may include, when present, less than 5 wt. % second promoter based on the total weight of the catalyst. For example, the catalyst may include from greater than 0 wt. % to 5 wt. %, from greater than 0 wt. % to 4 wt. %, from greater than 0 wt. % to 3 wt. %, from greater than 0 wt. % to 2 wt. %, from greater than 0 wt. % to 1 wt. %, from 1 wt. % to 5 wt. %, from 1 wt. % to 4 wt. %, from 1 wt. % to 3 wt. %, from 1 wt. % to 2 wt. %, from 2 wt. % to 5 wt. %, from 2 wt. % to 4 wt. %, from 2 wt. % to 3 wt. %, from 3 wt. % to 5 wt. %, from 3 wt. % to 4 wt. %, or from 4 wt. % to 5 wt. % second promoter based on the total weight of the catalyst.

In one or more embodiments, the catalyst includes a support material. Specifically, the catalyst may include the active metal component, the first promoter component, and optionally the second promoter, disposed and/or dispersed on the support material. In some embodiments, the support material includes one or more of alumina, silica-containing alumina, titanium-containing alumina, lanthanide-containing alumina, zirconium-containing alumina, magnesia-containing alumina, and a combination of two or more thereof.

Referring still to the FIGURE, the dehydrogenated hydrocarbon effluent and the catalyst may be passed out of the downstream reactor section 230 to a separation device 220 in the catalyst separation section 210. The catalyst may be separated from the dehydrogenated hydrocarbon effluent in the separation device 220. The dehydrogenated hydrocarbon effluent may then be transported out of the catalyst separation section 210. For example, the separated dehydrogenated hydrocarbon effluent may be removed from the reactor system 102 via a pipe 420 at a gas outlet port 216 of the catalyst separation section 210. In one or more embodiments, the separation device 220 may be a cyclonic separation system, which may include two or more stages of cyclonic separation.

Referring still to the FIGURE, following separation from the dehydrogenated hydrocarbon effluent in the separation device 220, the catalyst may generally move through the stripper 224 to the reactor catalyst outlet port 222 where the catalyst may be transferred out of the reactor portion 200 via standpipe 426 and into the combustor 350 of the catalyst-processing portion 300. Optionally, the catalyst may also be transferred directly back into the upstream reactor section 250 via standpipe 422. In one or more embodiments, recycled catalyst from the stripper 224 may be premixed with processed catalyst from the catalyst processing portion 300 in the transport riser 430.

Once passed to the catalyst-processing portion 300, the catalyst may be processed in the catalyst-processing portion 300. As used in the present disclosure, the term “catalyst processing” refers to preparing the catalyst for re-introduction into the reactor portion of the reactor system. In one or more embodiments, processing the catalyst includes removing coke deposits from the catalyst, raising the temperature of the catalyst through combustion of a combustion fuel, reactivating the catalyst, stripping one or more constituents from the catalyst, or combinations of these.

In some embodiments, processing the catalyst includes at least one fuel gas and coke on the deactivated catalyst in the presence of oxygen in the combustor 350 to remove coke deposits on the catalyst and/or heat the catalyst to produce a processed catalyst and combustion gases. As used in the present disclosure, the term “processed catalyst” refers to catalyst that has been processed in the catalyst-processing portion 300 of the reactor system 102. The processed catalyst may be separated from the combustion gases in the catalyst separation portion 310 and, in some embodiments, may then be reactivated by conducting an oxygen treatment of the heated catalyst. The oxygen treatment may include contacting the catalyst with an oxygen-containing gas for a period of time sufficient to reactivate the catalyst.

In one or more embodiments, the combustion fuel includes coke or other contaminants deposited on the catalyst in the reactor portion 200. The catalyst may be coked following the reactions in the reactor portion 200, and the coke may be removed from the catalyst by a combustion reaction in the combustor 350. For example, an oxidizer (such as air) may be fed into the combustor 350 via the air inlet 428. Alternatively or additionally, such as when coke is not formed on the catalyst or an amount of coke formed on the catalyst is not sufficient to burn off to heat the catalyst to a desired temperature, a supplemental fuel may be injected into the combustor 350, which may be burned to heat the catalyst. Suitable supplemental fuels may include methane, natural gas, ethane, propane, hydrogen, or any gas that provides energy value upon combustion. In one or more embodiments, the catalyst may be only lightly coked, and in these embodiments, the supplemental fuel is the primary fuel used to heat the catalyst.

The processed catalyst may be passed out of the combustor 350 and through the riser 330 to a riser termination separator 378, where the gas and solid components from the riser 330 may be at least partially separated. The vapor and remaining solids may be transported to a secondary separation device 320 in the catalyst separation section 310 where the remaining processed catalyst is separated from the gases from the catalyst processing (e.g., gases emitted by combustion of coke deposits and supplemental fuel). In some embodiments, the secondary separation device 320 may include one or a plurality of cyclone separation units, which may be arranged in series or in multiple cyclone pairs. The combustion gases from combustion of coke and/or the supplemental fuel during processing of the catalyst or other gases introduced to the catalyst during catalyst processing may be removed from the catalyst-processing portion 300 via a combustion gas outlet 432.

As previously discussed, processing the catalyst in the catalyst-processing portion 300 of the reactor system 102 may include reactivating the catalyst. Combustion of the supplemental fuel in the presence of the catalyst to heat the catalyst may further deactivate the catalyst. Accordingly, in some embodiments, the catalyst may be reactivated by conditioning the catalyst through an oxygen treatment. The oxygen treatment to reactivate the catalyst may be conducted after combustion of the supplemental fuel to heat the catalyst. In some embodiments, the oxygen treatment includes treating the processed catalyst with an oxygen-containing gas. The oxygen-containing gas may include an oxygen content of from 5 mole percent (mol. %) to 100 mol. % based on total molar flow rate of the oxygen-containing gas. In some embodiments, the oxygen treatment includes maintaining the processed catalyst at a temperature of at least 660° C. while exposing the catalyst to a flow of an oxygen-containing gas for a period of time sufficient to reactivate the processed catalyst (e.g., increase the catalytic activity of the processed catalyst).

In one or more embodiments, treatment of the processed catalyst with the oxygen-containing gas is conducted in the oxygen treatment zone 370. In some embodiments, the oxygen treatment zone 370 is downstream of the catalyst separation portion 310 of the catalyst-processing portion 300, such that the processed catalyst is separated from the combustion gases before being exposed to the oxygen-containing gas during the oxygen treatment. In some embodiments, the oxygen treatment zone 370 includes a fluid solids contacting device. The fluid solids contacting device may include baffles or grid structures to facilitate contact of the processed catalyst with the oxygen-containing gas. Examples of fluid solid contacting devices are described in further detail in U.S. Pat. Nos. 9,827,543 and 9,815,040, the contents of both of which are incorporated herein by reference.

In one or more embodiments, processing the catalyst in the catalyst-processing portion 300 of the reactor system 102 includes stripping the processed catalyst of molecular oxygen trapped within or between catalyst particles and physisorbed oxygen that is desorbable at a temperature of at least 660° C. The stripping step may include maintaining the processed catalyst at a temperature of at least 660° C. and exposing the processed catalyst to a stripping gas that is substantially free of molecular oxygen and combustible fuels for a period of time sufficient to remove the molecular oxygen from between particles and physisorbed oxygen that is desorbable at the temperature of at least 660° C. Further description of these catalyst reactivation processes are disclosed in U.S. Pat. No. 9,834,496, the entire content of which is incorporated herein by reference.

Referring still to the FIGURE, following processing of the catalyst, the processed catalyst may be passed from the catalyst-processing portion 300 back into the reactor portion 200 via standpipe 424. For example, the processed catalyst may be passed from the oxygen treatment zone 370 to the upstream reactor section 250 via standpipe 424 and transport riser 430, where the processed catalyst may be further utilized in a dehydrogenation reaction of a hydrocarbon-containing feed. Accordingly, in operation, the catalyst may cycle between the reactor portion 200 and the catalyst-processing portion 300. In general, the processed chemical streams, including the hydrocarbon-containing feed and the dehydrogenated hydrocarbon effluent may be gaseous, and the catalyst may be a fluidized particulate solid. In one or more embodiments, the reactor system 102 may include a hydrogen inlet stream 480 which provides supplemental hydrogen to the reactor system 102.

As discussed previously, combustion reactions in the combustor 350 (i.e., the combustion of the combustion fuel) may be promoted by the catalyst. That is, the catalyst may provide combustion activity in the combustor 350. However, the combustion activity of the catalyst may decrease over time as the catalyst is cycled between the reactor portion 200 and the catalyst-processing portion 300. As a result, during operation of the reactor system 102, the combustion fuel may no longer combust at the typical operating temperatures and pressures of the combustor 350 without sufficient maintenance of combustion activity in the combustor 350. Typical operating temperatures of the combustor 305 maybe from 600° C. to 850° C., and typical operating pressures of the combustor 350 may be from 15 pounds per square inch absolute (psia) to 60 psia.

In embodiments, the operating temperature of the combustor 305 may be from 50° C. to 200° C. greater than the temperature at which the dehydrogenation is performed. For instance, the combustion temperature may be from 60° C. to 200° C. greater, such as from 70° C. to 200° C., from 80° C. to 200° C., from 90° C. to 200° C., from 100° C. to 200° C., from 110° C. to 200° C., from 120° C. to 200° C., from 130° C. to 200° C., from 140° C. to 200° C., from 150° C. to 200° C., from 160° C. to 200° C., from 170° C. to 200° C., from 180° C. to 200° C., from 190° C. to 200° C., from 50° C. to 190° C., from 50° C. to 180° C., from 50° C. to 170° C., from 50° C. to 160° C., from 50° C. to 150° C., from 50° C. to 140° C., from 50° C. to 130° C., from 50° C. to 120° C., from 50° C. to 110° C., from 50° C. to 100° C., from 50° C. to 90° C., from 50° C. to 80° C., from 50° C. to 70° C., or from 50° C. to 60° C. greater than the temperature at which the dehydrogenation is performed. As described above, the presence of the second noble metal in the promoter component is believed to reduce the temperature required for combusting the catalyst after the dehydrogenation stage. As a result, the two components of the reactor system, the reactor portion and the catalyst-processing portion 300, may be operated more efficiently, giving the operator a higher level of control over the thermodynamics of the process.

According to an aspect, either alone or in combination with any other aspect, a method for dehydrogenation of one or more hydrocarbons and regeneration and reactivation of a catalyst composition includes contacting a first gaseous stream comprising a first hydrocarbon with a catalyst composition in a dehydrogenation reactor at a first temperature, thereby producing a first dehydrogenated hydrocarbon and a deactivated catalyst composition; combusting at least one fuel gas and coke on the deactivated catalyst in the presence of oxygen at a second temperature, thereby producing a heated catalyst composition; and reactivating the catalyst in the presence of oxygen. The second temperature is from 50° C. to 200° C. greater than the first temperature. The catalyst composition includes an active metal comprising gallium, a support, and a promoter comprising platinum and at least one noble metal selected from the group consisting of ruthenium, rhodium, palladium, rhenium, iridium, and a combination of two or more thereof. The ratio of total second noble metal to platinum by weight is from 0.05 to 1.5.

According to a second aspect, either alone or in combination with any other aspect, the first hydrocarbon is ethane and the first temperature is from 700° C. to 850° C.

According to a third aspect, either alone or in combination with any other aspect, the first hydrocarbon is propane and the first temperature is from 550° C. to 700° C.

According to a fourth aspect, either alone or in combination with any other aspect, the first hydrocarbon is isobutane and the first temperature is from 500° C. to 650° C.

According to a fifth aspect, either alone or in combination with any other aspect, the method further includes contacting a second gaseous stream with the catalyst composition after the reactivating, wherein the second gaseous stream comprises a second hydrocarbon different from the first hydrocarbon, thereby producing a second dehydrogenated hydrocarbon and the deactivated catalyst composition.

According to a sixth aspect, either alone or in combination with any other aspect, the dehydrogenation reactor comprises a fluidized bed.

According to a seventh aspect, either alone or in combination with any other aspect, the fuel gas comprises methane.

According to an eighth aspect, either alone or in combination with any other aspect, the noble metal is selected from the group consisting of ruthenium, rhodium, palladium, iridium, and a combination of two or more thereof.

According to a ninth aspect, either alone or in combination with any other aspect, the noble metal is palladium.

According to a tenth aspect, either alone or in combination with any other aspect, a catalyst composition includes an active metal comprising gallium, a support, and a promoter comprising platinum and at least one noble metal selected from the group consisting of ruthenium, rhodium, palladium, rhenium, iridium, and a combination of two or more thereof. The ratio of total second noble metal to platinum by weight is from 0.05 to 1.5.

According to an eleventh aspect, either alone or in combination with any other aspect, the support is selected from the group consisting of alumina, silica-containing alumina, titanium-containing alumina, lanthanide-containing alumina, zirconium-containing alumina, magnesia-containing alumina, and a combination of two or more thereof.

According to a twelfth aspect, either alone or in combination with any other aspect, the catalyst composition comprises from 0.1 wt % to 10 wt % active metal component.

According to a thirteenth aspect, either alone or in combination with any other aspect, the catalyst composition comprises from 5 parts per million by weight (ppmw) to 500 ppmw platinum.

According to a fourteenth aspect, either alone or in combination with any other aspect, the catalyst composition further comprises a second promoter selected from the group consisting of an alkali metal, an alkaline earth metal, and a combination of the alkali metal and the alkaline earth metal.

According to a fifteenth aspect, either alone or in combination with any other aspect, the catalyst composition comprises from greater than 0 wt % to 5 wt % second promoter.

One or more features of the present disclosure are illustrated in view of the examples as follows:

### EXAMPLES

The following examples are illustrative in nature and should not serve to limit the scope of the present application.

### Example 1—Dehydrogenation and Combustion with Platinum- and Palladium-Loaded Gallium-Based Catalysts

A series of alumina supported catalysts are made using a conventional incipient wetness method. Incipient wetness methods are conducted by first dissolving the metal precursor, i.e., gallium nitrate, potassium nitrate, tetraamineplatinum nitrate, tetraamine palladium nitrate, in water. The resulting solution is contacted with a catalyst support, i.e., alumina, having the same pore volume as the volume of solution added overnight, i.e. over a period of time ranging from 6 hours to 14 hours. The equivalent volume promotes a capillary action uptake of the metal instead of a diffusion process, which is much slower than the capillary action process. The resulting platinum- and palladium-loaded gallium-based catalysts are dried and calcined at 750° C. for 2 hours. All catalysts have the same Ga and K loading (1.5 wt. % and 0.25 wt %, respectively). The Pt and Pd loading of each is provided in Table 1.

TABLE 1
Pt—Pd-loaded gallium based catalyst compositions

| | Pt loading | Pd loading |
|---|---|---|
| Sample | (ppmw) | (ppmw) |
| A (Comparative) | 80 | 0 |
| B | 80 | 20 |
| C | 80 | 40 |
| D | 80 | 100 |
| E | 80 | 200 |
| F (Comparative) | 0 | 200 |
| G (Comparative) | 200 | 0 |
| H | 200 | 40 |
| I | 200 | 100 |

The dehydrogenation performance of these platinum- and palladium-loaded gallium-based catalysts is illustrated using propane dehydrogenation as a model reaction. The objective is to determine the range of Pd addition which has no or little impact on dehydrogenation performance.

Dehydrogenation performance was evaluated in a fixed bed lab testing rig under ambient pressure using reaction-regeneration cycles. Each cycle includes a dehydrogenation step with a 120 second propane pulse (95% propane/5% inert gas) at a temperature of 625° C. and a weight hourly space velocity (WHSV) of the propane of 8 hr⁻¹, and a regeneration step at T_{Regen} of 730° C., where the catalyst is first treated for 3 minutes with a simulated combustion effluent (8% CO₂, 4.0% O₂, 16% H₂O in inert gas), followed by 10 minutes in air. Samples are collected at cycle 20 for about 17 seconds.

The composition of the reaction products was determined by Gas Chromatography (GC). The feed conversion and product selectivity were determined by equations (1) and (2):

$$\\ \begin{matrix}Propane Conversion = \frac{{\sum }_{k} {n}_{k} {C}_{k}}{{n}_{C 3} {C}_{C 3} + {\sum }_{k} {n}_{k} {C}_{k}} & ( 1 )\end{matrix}$$

$$\\ \begin{matrix}Propylene Selectivity = \frac{{n}_{C 3 =} {n}_{C 3 =}}{{\sum }_{k} {n}_{k} {C}_{k}} & ( 2 )\end{matrix}$$

in which:
k refers to Product k, and the products analyzed include methane, ethane, ethylene, propylene, species including four carbon atoms (C4s), species including five carbon atoms (C5s), and species including six or more carbon atoms (C6s+).
nₖ refers to the number of carbons in the chemical formula of Product k; n_{C3}-refers to the number of carbons in the specific product, propylene; while n_{C3} refers to the number of carbons in chemical formula of reactant propane; both n_{C3=} and n_{C3} are equal to 3.
Cₖ refers to molar fraction of general Product K in the reaction effluent, and C_{C3=} refers to the molar fraction of specific product, propylene, in the reaction effluent; while C_{C3} refers to the molar fraction of unreacted reactant propane, in the reaction effluent.

The results, which are summarized in Table 2, indicate nearly equivalent dehydrogenation performance at low Pd level (Pd:Pt ratio <50%, Samples B and C versus Sample A) and acceptable dehydrogenation performance with higher Pd level (Pd:Pt ratio <150%, Samples D and E versus Sample A). Sample F, having only Pd and no Pt, exhibited very poor dehydrogenation performance.

TABLE 2
Pt—Pd-loaded gallium based catalyst
dehydrogenation performance

| | % Propane | % Selectivity |
|---|---|---|
| Sample | Conversion | for Propylene |
| A (Comparative) | 55 | 96.4 |
| B | 53 | 96.5 |
| C | 53.4 | 96.3 |
| D | 49.6 | 96.1 |
| E | 45.6 | 96.4 |
| F (Comparative) | 26.3 | 91.6 |

A similar trend was observed when increasing Pt loading in the catalyst, as shown in Table 3. For these experiments, the protocol described above is used except the WHSV of the propane is increased to 10 hr⁻¹.

TABLE 3
Pt—Pd-loaded gallium based catalyst
dehydrogenation performance

| | % Propane | % Selectivity |
|---|---|---|
| Sample | Conversion | for Propylene |
| G (Comparative) | 54.5 | 96.3 |
| H | 54.6 | 96.6 |
| I | 50.6 | 96.3 |

A fuel gas combustion test was carried out in a fixed bed lab reactor at ambient pressure with WHSV of methane of 0.59 hr⁻¹. The catalyst is heated under a flow of nitrogen to 540° C. Subsequently, the nitrogen is replaced by air for 2 minutes before introducing methane into the feed with a targeted composition of 2 volume % (vol. %) CH₄ in air. The temperature is increased stepwise with a ramp rate of 10° C./min and allowed to remain at targeted reaction temperatures for 5.25 minutes, until a temperature of 800° C. is reached. Gas Chromatography (GC) samples of the effluent are taken during each 5.25 minute temperature dwell step. The condition in the lab is selected to facilitate differentiation of catalyst performance. In the pilot scale and commercial scale, fuel gas combustion would need to be complete (100% conversion) or nearly complete so that the fuel gas in effluent is well below the Lower Explosive or Flammable Limit, as required for safe operation. The results are shown in Table 4.

TABLE 4
Pt—Pd-loaded gallium based catalyst
methane combustion performance

CH₄ Conversion at reaction temperature

| Sample | 625° C. | 650° C. | 725° C. | 750° C. |
|---|---|---|---|---|
| A (Comparative) | 7.9 | 10.5 | 36.4 | 41.4 |
| B | 14.8 | 16.6 | 34 | 41.7 |
| C | 24.7 | 26.4 | 34.9 | 40.1 |
| D | 38.5 | 39.3 | 41.3 | 43.6 |
| G (Comparative) | 2.6 | 9.5 | 34 | 44.5 |

When the temperature is above 720° C., a catalyst without Pd can provide good methane combustion activity. However, when the temperature is less than 720° C., the presence of Pd enhances combustion activity of catalysts. Compare, for instance, the performance of Sample D versus that of Sample A. Thus, a combination of Pd and Pt as a promoter for fuel gas combustion allows fuel gas combustion in a broader temperature window.

### Example 2—Dehydrogenation and Combustion with Platinum- and Iridium-Loaded Gallium-Based Catalysts

Pt—Ir-loaded gallium based catalyst samples are made using the same procedure described above for the Pt—Pd-loaded gallium based catalyst, except Ir is loaded in a second impregnation step to Catalyst A using iridium nitrate. The compositions are provided in Table 5.

TABLE 5
Pt—Ir-loaded gallium based catalyst compositions

| | Pt loading | Ir loading |
|---|---|---|
| Sample | (ppmw) | (ppmw) |
| A (Comparative) | 80 | 0 |
| J | 80 | 40 |
| K | 80 | 100 |

The same combustion analysis discussed above as the Pt—Pd-loaded gallium based catalysts is performed using the Pt—Ir-loaded gallium based catalysts. The results are provided in Table 6.

TABLE 6
Pt—Ir-loaded gallium based catalyst
methane combustion performance

CH₄ Conversion at reaction temperature

| Sample | 625° C. | 650° C. | 725° C. | 750° C. |
|---|---|---|---|---|
| A (Comparative) | 7.9 | 10.5 | 36.4 | 41.4 |
| J | 13.1 | 14.2 | 33.2 | 42.2 |
| K | 14.5 | 16.9 | 33.1 | 40.7 |

Similar to the results with the Pt—Pd-loaded gallium based catalysts, when the temperature is above 720° C., a catalyst without Ir can provide good methane combustion activity. However, when the temperature is less than 720° C., the presence of Ir enhances combustion activity of catalysts. Compare, for instance, the performance of Sample K versus that of Sample A. Thus, a combination of Ir and Pt as a promoter for fuel gas combustion allows fuel gas combustion in a broader temperature window.

### Example 3—Dehydrogenation and Combustion with Platinum- and Ruthenium-Loaded Gallium-Based Catalysts

Pt—Ru-loaded gallium based catalyst samples are made using the same procedure described above for the Pt—Pd-loaded gallium based catalyst, except Ru is loaded in a second impregnation step to Catalyst A using ruthenium chloride. The compositions are provided in Table 7.

TABLE 7
Pt—Ru-loaded gallium based catalyst compositions

| | Pt loading | Ru loading |
|---|---|---|
| Sample | (ppmw) | (ppmw) |
| A (Comparative) | 80 | 0 |
| L | 80 | 100 |

The same combustion analysis discussed above as the Pt—Pd-loaded gallium based catalysts is performed using the Pt—Ru-loaded gallium based catalysts. The results are provided in Table 8.

TABLE 8
Pt—Ru-loaded gallium based catalyst
methane combustion performance

CH₄ Conversion at reaction temperature

| Sample | 625° C. | 650° C. | 725° C. | 750° C. |
|---|---|---|---|---|
| A (Comparative) | 7.9 | 10.5 | 36.4 | 41.4 |
| L | 27.1 | 37.9 | 76.3 | 85.6 |

Similar to the results with the Pt—Pd-loaded gallium based catalysts, when the temperature is above 720° C., a catalyst without Ru can provide good methane combustion activity. However, when the temperature is less than 720° C., the presence of Ru enhances combustion activity of catalysts. Compare, for instance, the performance at 650° C. of Sample L versus that of Sample A. Thus, a combination of Ru and Pt as a promoter for fuel gas combustion allows fuel gas combustion in a broader temperature window.

### Example 4—Dehydrogenation and Combustion with Platinum- and Rhodium-Loaded Gallium-Based Catalysts

Pt—Rh-loaded gallium based catalyst samples are made using the same procedure described above for the Pt—Pd-loaded gallium based catalyst, except Rh is loaded in a second impregnation step to Catalyst A using rhodium nitrate. The compositions are provided in Table 9.

TABLE 9
Pt—Rh-loaded gallium based catalyst compositions

| | Pt loading | Rh loading |
|---|---|---|
| Sample | (ppmw) | (ppmw) |
| A (Comparative) | 80 | 0 |
| M | 80 | 100 |

The same combustion analysis discussed above as the Pt—Pd-loaded gallium based catalysts is performed using the Pt—Rh-loaded gallium based catalysts. The results are provided in Table 10.

TABLE 10
Pt—Rh-loaded gallium based catalyst
methane combustion performance

CH₄ conversion at reaction temperature

| Sample | 625° C. | 650° C. | 725° C. | 750° C. |
|---|---|---|---|---|
| A (Comparative) | 7.9 | 10.5 | 36.4 | 41.4 |
| M | 56 | 62 | 75.2 | 74.3 |

Similar to the results with the Pt—Pd-loaded gallium based catalysts, when the temperature is above 720° C., a catalyst without Rh can provide good methane combustion activity. However, when the temperature is less than 720° C., the presence of Rh enhances combustion activity of catalysts. Compare, for instance, the performance at 650° C. of Sample M versus that of Sample A. Thus, a combination of Rh and Pt as a promoter for fuel gas combustion allows fuel gas combustion in a broader temperature window.

It should be apparent to those skilled in the art that various modifications can be made to the described embodiments without departing from the spirit and scope of the claimed subject matter. Thus, it is intended that the specification cover modifications and variations of the described embodiments provided such modification and variations come within the scope of the appended claims and their equivalences.

1. A method for dehydrogenation of one or more hydrocarbons and regeneration and reactivation of a catalyst composition, the method comprising:
contacting a first gaseous stream comprising a first hydrocarbon with a catalyst composition in a dehydrogenation reactor at a first temperature, thereby producing a first dehydrogenated hydrocarbon and a deactivated catalyst composition;
combusting at least one fuel gas and coke on the deactivated catalyst in the presence of oxygen at a second temperature, thereby producing a heated catalyst composition; and
reactivating the catalyst in the presence of oxygen,
wherein:
the second temperature is from 50° C. to 200° C. greater than the first temperature; and
the catalyst composition comprises:
an active metal comprising gallium;
a promoter comprising platinum and at least one noble metal selected from the group consisting of ruthenium, rhodium, palladium, rhenium, iridium, and a combination of two or more thereof, a ratio of total second noble metal to platinum by weight being from 0.05 to 1.5, and
a support.

2. The method of claim 1, wherein the first hydrocarbon is ethane and the first temperature is from 700° C. to 850° C.

3. The method of claim 1, wherein the first hydrocarbon is propane and the first temperature is from 550° C. to 700° C.

4. The method of claim 1, wherein the first hydrocarbon is isobutane and the first temperature is from 500° C. to 650° C.

5. The method of claim 1, further comprising:
contacting a second gaseous stream with the catalyst composition after the reactivating, wherein the second gaseous stream comprises a second hydrocarbon different from the first hydrocarbon, thereby producing a second dehydrogenated hydrocarbon and the deactivated catalyst composition.

6. The method of claim 1, wherein the dehydrogenation reactor comprises a fluidized bed.

7. The method of claim 1, wherein the fuel gas comprises methane.

8. The method of claim 1, wherein the noble metal is selected from the group consisting of ruthenium, rhodium, palladium, iridium, and a combination of two or more thereof.

9. The method of claim 1, wherein the noble metal is palladium.

10. A catalyst composition comprising:
an active metal component comprising gallium;
a promoter component comprising platinum and at least one noble metal selected from the group consisting of ruthenium, rhodium, palladium, rhenium, iridium, and a combination of two or more thereof; and
a support,
wherein a ratio of total second noble metal to platinum by weight is from 0.05 to 1.5.

11. The catalyst composition of claim 10, wherein the support is selected from the group consisting of alumina, silica-containing alumina, titanium-containing alumina, lanthanide-containing alumina, zirconium-containing alumina, magnesia-containing alumina, and a combination of two or more thereof.

12. The catalyst composition of claim 10, wherein the catalyst composition comprises from 0.1 wt % to 10 wt % active metal component.

13. The catalyst composition of claim 10, wherein the catalyst composition comprises from 5 parts per million by weight (ppmw) to 500 ppmw platinum.

14. The catalyst composition of claim 10, wherein the catalyst composition further comprises a second promoter selected from the group consisting of an alkali metal, an alkaline earth metal, and a combination of the alkali metal and the alkaline earth metal.

15. The catalyst composition of claim 14, wherein the catalyst composition comprises from greater than 0 wt % to 5 wt % second promoter.
